SEQUENCE LISTING (27 sequences)

SEQ ID NO: 1 - DNA, 15 bp, synthetic construct
  source 1..15: mol_type = other DNA
ccagcagggggcgct

SEQ ID NO: 2 - DNA, 15 bp, synthetic construct
  source 1..15: mol_type = other DNA
cgaggaggggacgct

SEQ ID NO: 3 - DNA, 15 bp, synthetic construct
  source 1..15: mol_type = other DNA
caagcgtggtgcgct

SEQ ID NO: 4 - DNA, 15 bp, synthetic construct
  source 1..15: mol_type = other DNA
cgagcgtggtgcgct

SEQ ID NO: 5 - DNA, 20 bp, synthetic construct
  source 1..20: mol_type = other DNA
tttggtgcatgctccaggcg

SEQ ID NO: 6 - DNA, 31 bp, synthetic construct
  source 1..31: mol_type = other DNA
gtcagcgccccctgctggcctggagcatgca

SEQ ID NO: 7 - DNA, 31 bp, synthetic construct; identical to SEQ ID NO: 6
  source 1..31: mol_type = other DNA

SEQ ID NO: 8 - DNA, 20 bp, synthetic construct
  source 1..20: mol_type = other DNA
cctccaaccgtcccctccag

SEQ ID NO: 9 - DNA, 20 bp, synthetic construct
  source 1..20: mol_type = other DNA
ggatgagagaagagagtgct

SEQ ID NO: 10 - DNA, 65 bp, synthetic construct
  source 1..65: mol_type = other DNA
attgggctggcgtgttcagccaggaaactgcctagcccagcagggggcgctactctcttctctca

SEQ ID NO: 11 - DNA, 65 bp, synthetic construct
  source 1..65: mol_type = other DNA
attgggctggcgtgttcagccaggaaactgcctagcagcgccccctgctggactctcttctctca

SEQ ID NO: 12 - DNA, 20 bp, synthetic construct
  source 1..20: mol_type = other DNA
gttgatgacaatatagtttg

SEQ ID NO: 13 - DNA, 20 bp, synthetic construct
  source 1..20: mol_type = other DNA
ccctctcttcgcgcaggcct

SEQ ID NO: 14 - DNA, 60 bp, synthetic construct
  source 1..60: mol_type = other DNA
aggcgtcccggcgctaggagggacgcacccaggccagcagggggcgctcctgcgcgaaga

SEQ ID NO: 15 - DNA, 60 bp, synthetic construct
  source 1..60: mol_type = other DNA
aggcgtcccggcgctaggagggacgcacccaggagcgccccctgctggcctgcgcgaaga

SEQ ID NO: 16 - DNA, 20 bp, synthetic construct
  source 1..20: mol_type = other DNA
ctgcatttagggattctccg

SEQ ID NO: 17 - DNA, 20 bp, synthetic construct
  source 1..20: mol_type = other DNA
gacatgttccctgagagcct

SEQ ID NO: 18 - DNA, 38 bp, synthetic construct
  source 1..38: mol_type = other DNA
ggagctgggtagcgccccctgctggctctcagggaaca

SEQ ID NO: 19 - DNA, 52 bp, synthetic construct
  source 1..52: mol_type = other DNA
acgtcaccagctggagctgggtccagcagggggcgctctctcagggaacatg

SEQ ID NO: 20 - DNA, 20 bp, synthetic construct
  source 1..20: mol_type = other DNA
agcagaatcaggcttaaatc

SEQ ID NO: 21 - DNA, 20 bp, synthetic construct
  source 1..20: mol_type = other DNA
ggaaaaagttaagcagaatc

SEQ ID NO: 22 - DNA, 15 bp, synthetic construct
  source 1..15: mol_type = other DNA
cggggaggggaggct

SEQ ID NO: 23 - DNA, 15 bp, synthetic construct
  source 1..15: mol_type = other DNA
agcgccccctgctgg

SEQ ID NO: 24 - DNA, 15 bp, synthetic construct
  source 1..15: mol_type = other DNA
cctggggggtgggga

SEQ ID NO: 25 - DNA, 15 bp, synthetic construct
  source 1..15: mol_type = other DNA
gttggagggggcgag

SEQ ID NO: 26 - DNA, 15 bp, synthetic construct
  source 1..15: mol_type = other DNA
caacctcccccgctc

SEQ ID NO: 27 - DNA, 15 bp, synthetic construct
  source 1..15: mol_type = other DNA
tcgcgggggacgacc

(19) United States
(12) Patent Application Publication
(10) Pub. No.: US 2026/0043049 A1
(43) Pub. Date: Feb. 12, 2026

(54) CCCTC-BINDING FACTOR (CTCF)-MEDIATED GENE ACTIVATION

(71) Applicant: The General Hospital Corporation, Boston, MA (US)

(72) Inventors: J. Keith Joung, Winchester, MA (US); Yugyoung Esther Tak, Charlestown, MA (US); Rebecca Tayler Cottman, Cambridge, MA (US)

(21) Appl. No.: 18/998,936

(22) PCT Filed: Jul. 24, 2023
(86) PCT No.: PCT/US2023/070852

§ 371 Date: Jan. 27, 2025

Related U.S. Application Data
(60) Provisional application No. 63/392,065, filed on Jul. 25, 2022.

(51) Int. Cl. C12N 15/90; C12N 9/22; C12N 15/11

(52) U.S. Cl. CPC C12N 15/907; C12N 9/222; C12N 15/11; C12N 2310/20

(57) ABSTRACT

Methods for increasing expression of a target gene, the method comprising introducing a CCCTC-binding factor (CTCF) binding site (CTCF-BS) into a promoter region of the target gene, e.g., within 500, 250, 200, 150, 100, 50, or 25 nucleotides of the transcription start site (TSS) for the target gene, and optionally expressing in or introducing into the cell a CTCF protein or variant thereof.

### CLAIM OF PRIORITY

This application claims the benefit of U.S. Provisional Patent Application Ser. No. 63/392,065, filed on Jul. 25, 2022. The entire contents of the foregoing are hereby incorporated by reference.

### FEDERALLY SPONSORED RESEARCH OR DEVELOPMENT

This invention was made with Government support under Grant Nos. GM118158 and HG009490 awarded by the National Institutes of Health. The Government has certain rights in the invention.

### SEQUENCE LISTING

This application contains a Sequence Listing that has been submitted electronically as an XML file named “29539-0691WO1_SL_ST26.XML.” The XML file, created on Jul. 20, 2023, is 24,958 bytes in size. The material in the XML file is hereby incorporated by reference in its entirety.

### TECHNICAL FIELD

Methods for increasing expression of a target gene, the method comprising introducing a CCCTC-binding factor (CTCF) binding site (CTCF-BS) into a promoter region of the target gene, e.g., within 1000, 500, 250, 200, 150, 100, 50, 25, or 10 nucleotides of the transcription start site (TSS) for the target gene, and optionally expressing in or introducing into the cell a CTCF protein or variant thereof.

### BACKGROUND

Human gene expression is known to be regulated by multiple transcription factors and coactivators that are recruited to promoter and enhancer regulatory sequences. Promoter sequences are located close to the transcription start site (TSS). By contrast, enhancer elements exert their effects over long linear distances across the genome, a phenomenon enabled by three-dimensional (3D) folding of the genome that can bring these enhancers into proximity with promoters.

### SUMMARY

Provided herein are methods of increasing expression of a target gene in a cell. The methods comprise introducing a canonical CCCTC-binding factor (CTCF) binding site (CTCF-BS) into a promoter region of the target gene, e.g., within 1000, 500, 250, 200, 150, 100, 50, 25, or 10 nucleotides of the transcription start site (TSS) for the target gene, optionally wherein the cell expresses a CTCF protein, optionally an endogenous CTCF protein. In some embodiments, the canonical CTCF-BS comprises the following core sequence: 5′-CCAGCAGGGGGCGCT-3′ (SEQ ID NO: 1). In some embodiments, the canonical CTCF-BS is introduced in the “right” orientation, i.e., in the sense strand with respect to the target gene. In some embodiments, the CTCF-BS is introduced into the target promoter using gene editing nucleases mediating non-homologous end-joining repair, capture of double-stranded oligonucleotides (dsODNs), or microhomology-mediated repair; prime editing; CRISPR-based editing; base editing; and homologous recombination or homology-directed repair.

In some embodiments, the cell expresses a CTCF protein, optionally an endogenous CTCF protein, or the methods include expressing in or introducing into the cell the CTCF protein. The CTCF can be, e.g., expressed from an endogenous CTCF gene, or stably or transiently expressed or overexpressed from an exogenously added CTCF sequence.

Also provided herein are methods for increasing expression of a target gene that comprise introducing a non-canonical CCCTC-binding factor (CTCF) binding site (CTCF-BS) into a promoter region of the target gene, e.g., within 1000, 500, 250, 200, 150, 100, 50, 25, or 10 nucleotides of the transcription start site (TSS) for the target gene, and expressing (e.g., stably or transiently expressing) in or introducing into the cell a variant CTCF protein with an altered DNA-binding specificity that binds the non-canonical CTCF-BS. In some embodiments, the non-canonical CTCF-BS comprises one of the following core sequences: 5′-CGAGGAGGGGACGCT-3′ (SEQ ID NO: 2), 5′-CAAGCGTGGTGCGCT-3′ (SEQ ID NO:3), or 5′-CGAGCGTGGTGCGCT-3′ (SEQ ID NO:4). In some embodiments, the canonical or non-canonical CTCF-BS is introduced in the “right” orientation, i.e., in the sense strand with respect to the target gene. In some embodiments, non-canonical CTCF-BS is introduced into the target promoter using gene editing nucleases mediating non-homologous end-joining repair, capture of double-stranded oligonucleotides (dsODNs), or microhomology-mediated repair; prime editing; CRISPR-based editing; base editing; or homologous recombination or homology-directed repair.

In some embodiments of the methods described herein, the cell is in vitro. In some embodiments of the methods described herein, the cell is in a living animal, e.g., a mammal (e.g., a non-human mammal or a human).

Also provided herein are cells, e.g., isolated cells, comprising an exogenous canonical or non-canonical CCCTC-binding factor (CTCF) binding site (CTCF-BS) in a promoter region of a target gene in a cell, wherein expression of the target gene is increased with respect to a cell of the same type that does not comprise an exogenous CTCF-BS in the promoter region. In some embodiments, the exogenous canonical or non-canonical CTCF-BS is within 1000, 500, 250, 200, 150, 100, 50, 25, or 10 nucleotides of the transcription start site (TSS) for the target gene. In some embodiments, the isolated cells express an endogenous CTCF that binds the canonical CTCF-BS or a variant CTCF protein with an altered DNA-binding specificity that binds the non-canonical CTCF-BS. In some embodiments, the canonical CTCF-BS comprises the sequence: 5′-CCAGCAGGGGGCGCT-3′ (SEQ ID NO:1), or the non-canonical CTCF-BS comprises one of: 5′-CGAGGAGGGGACGCT-3′ (SEQ ID NO: 2), 5′-CAAGCGTGGTGCGCT-3′ (SEQ ID NO:3), or 5′-CGAGCGTGGTGCGCT-3′ (SEQ ID NO:4).

In some embodiments, the exogenous canonical or non-canonical CTCF-BS is present in the sense strand with respect to the target gene. In some embodiments the CTCF-BS is introduced into the target promoter using gene editing nucleases mediating non-homologous end-joining repair, capture of double-stranded oligonucleotides (dsODNs), or microhomology-mediated repair; prime editing; CRISPR-based editing; base editing; and homologous recombination or homology-directed repair.

In some embodiments the isolated cell is in vitro, or is in a living animal, e.g., a mammal (e.g., a non-human mammal or a human).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-E. Introduction of consensus CTCF binding sites (CBSs, also referred to herein as CTCF-BSs) by creating multiple nucleotide substitutions at the human SGCA promoter leads to transcriptional activation of this gene in K562 cells.
(A) SGCA RNA expression in K562 with exogenous expression of vCTCF.
(B) Schematic of the SGCA promoter region harboring non-CBS (SEQ ID NO: 22), located 70 bp upstream of the SGCA TSS.
(C)-(D) Schematics of sequence changes introduced into the non-CBS sequence (the off-target binding site for vCTCF) to create consensus CBSs in two different directions. (C), SEQ ID NOs: 22 and 1; (D), SEQ ID NOs: and 22 and 23.
(E) Introduction of a consensus CBS into the SGCA promoter leads to activation of SGCA expression in human K562 cells. K562 single cells clone with different editing frequencies (percentage values given in the x-axis legend) are shown on the x-axis. Clones labeled with T have a consensus CBS in the “right” direction (consensus CBS on the top strand) while those labeled with B have it in the “left” direction (consensus CBS on the bottom strand).

FIGS. 2A-B. Introduction of consensus CTCF binding sites (CBSs) by creating multiple nucleotide substitutions at the human SGCA promoter leads to transcriptional activation of this gene in HEK293T cells.
(A) DNA editing efficiencies and fold-activation of SGCA RNA transcript expression in HEK293T cell line clones isolated from CRISPR prime editing experiments intended to introduce a consensus CBS in the “right” or “>” orientation. Note that some clones (e.g., 1, 2) do not show any DNA editing (i.e., no introduction of a consensus CBS in any alleles) whereas others show variable levels of editing that presumably reflect whether one, two, or all three alleles in the cell clone were successfully edited. SEQ ID NOs: 22 and 1 are shown.
(B) Same as in (A) except that these clones were isolated from CRISPR prime editing experiments intended to introduce a consensus CBS in the “left” or “<” orientation. SEQ ID NOs; and 22 and 23 are shown.

FIG. 3. Endogenous CTCF binds to the consensus CBSs introduced at the SGCA promoter. CTCF ChIP followed by qPCR shows the enrichment of CTCF binding at the SGCA promoter in the HEK293T single-cell clonal lines that harbor the consensus CBS in the “right” and “left” orientations (clones 8 and 24, respectively). Note that clonal lines that do not harbor an introduced consensus CBS do not show CTCF enrichment at the SGCA promoter. The ZNF180 site and APOA1 site were used as positive and negative control sites, respectively, for CTCF binding in HEK293T.

FIGS. 4A-C. Introduction of consensus CTCF binding sites (CBSs) by creating multiple nucleotide substitutions at the human CD4 promoter leads to transcriptional activation of this gene in K562 cells.
(A) Schematic of the CD4 promoter region with the non-CBS sequence (located 35 bp upstream of the TSS) that we converted into a consensus CBS in the “right” or “left” orientation.
(B) Flow cytometry plots showing increased CD4 protein expression in K562 cells following electroporation of plasmid encoding CRISPR prime editor components needed to introduce the consensus CBSs in the “right” or “left” orientations. SEQ ID NOs: 24, 1, and 23 are shown.
(C) Quantitative RT-PCR experiments that quantify activation of CD4 RNA transcript expression in K562 cell clones harboring the consensus CBS introduced in the “right” or “left” orientation.

FIGS. 5A-B. Introduction of consensus CTCF binding sites (CBSs) by creating multiple nucleotide substitutions at the human HER2 promoter leads to transcriptional activation of this gene in K562 cells.
(A) Schematic of the HER2 promoter region with the non-CBS sequence (located 50 bp upstream of the TSS) that we converted into a consensus CBS in the “right” or “left” orientation. SEQ ID NOs: 25, 1, and 23 are shown.
(B) Flow cytometry plots showing increased HER2 protein expression in K562 cells following electroporation of plasmid encoding CRISPR prime editor components needed to introduce the consensus CBSs in the “right” or “left” orientations. SEQ ID NOs: 25, 1, 26, and 17 are shown (note the sequences in the right hand panel of FIG. 5B are presented in 3′→5′ orientation.

FIGS. 6A-B. Introduction of consensus CTCF binding sites (CBSs) by creating multiple nucleotide substitutions at the human IL2RA promoter leads to transcriptional activation of this gene in K562 cells.
(A) Schematic of the IL2RA promoter region with the non-CBS sequence (located 20 bp upstream of the TSS) that we converted into a consensus CBS in the “right” or “left” orientation. SEQ ID NOs. 1 and 23 are shown.
(B) Quantitative RT-PCR experiments that quantify activation of IL2RA RNA transcript expression in K562 cell clones harboring the consensus CBS introduced in the “right” or “left” orientation. We inserted a synthetic motif referred to as ELF (2×) as a positive control (previous experiment show that insertion of this sequence resulted in increased IL2RA expression.

FIG. 7. ChIP-seq data performed with anti-CTCF or anti-RAD21 antibodies for the SGCA locus in various clonal K562 lines. Two biological clonal lines for each of three different SGCA promoter sequences are shown (no introduced consensus CBS (wild-type), consensus CBS introduced in the “right” orientation, and consensus CBS introduced in the “left” orientation.

FIG. 8. ChIP-seq data performed with anti-H3K27Ac or anti-H3K4me3 antibodies for the SGCA locus in various clonal K562 lines. Two biological clonal lines for each of three different SGCA promoter sequences are shown (no introduced consensus CBS (wild-type), consensus CBS introduced in the “right” orientation, and consensus CBS introduced in the “left” orientation.

FIG. 9. HiChIP data performed with anti-CTCF antibody for the SGCA locus in K562 clonal lines. Two biological clonal lines for each of three different SGCA promoter sequences are shown (no introduced consensus CBS (wild-type), consensus CBS introduced in the “right” orientation, and consensus CBS introduced in the “left” orientation. Statistically significant CTCF loops are shown with the line thickness indicating the strength of interaction between the anchor points.

FIG. 10. Micro-C data for the SGCA locus in K562 clonal lines at 2 Kb resolution. One biological clonal line for each of the three different SGCA loci are shown (no introduced consensus CBS (wild type), consensus CBS introduced in the “right” orientation, and consensus CBS introduced in the “left” orientation. The dotted triangle on the left figure indicates a pre-existing TAD structure at SGCA locus. The TAD structure is maintained in the case of CBS introduced in the “right” orientation (middle figure) at the SGCA promoter, but the strength of the TAD is increased (shown as an arrow). CBS with the “left” orientation at the SGCA promoter strengths the sub TAD structures indicated in two dotted triangles.

FIGS. 11A-C. Transient transfection experiments using GFP reporter plasmids bearing various wild-type and edited SGCA, CD4, and HER2 promoter fragments
(A) Schematic of experimental details. Various size DNA fragments of the three different promoters either harboring or not harboring a consensus CBS were inserted upstream of a promoterless GFP reporter gene. These plasmids were Nucleofected into K562 cells and then assessed by flow cytometry for GFP expression and for RFP expression from a co-transfected plasmid that constitutively expresses RFP (and which serves as a control for transfection efficiency).
(B-C) GFP/RFP ratios (y-axis) determined by flow cytometry for cells transfected with the various GFP reporter plasmids harboring different promoter fragments (x-axis) and the control RFP plasmid.

### DETAILED DESCRIPTION

CTCF is a multi-zinc finger protein that has been shown to play a key role in establishing and maintaining the 3D architecture of the genome. It is believed to do so by binding to specific DNA sequences and mediating interactions with the cohesion complex to create topologically associated domains (TADs). Although CTCF is generally not believed to function directly as an activator or repressor of transcription, it has also been implicated in potentially mediating long-range enhancer-promoter interactions (Kubo et al., Nat Struct Mol Biol. 2021 February; 28 (2): 152-161; Oh et al., Nature. 2021 July; 595 (7869): 735-740; Ren et al., Mol Cell. 2017 Sep. 21; 67 (6):1049-1058.e6).

Epigenetic editing is a technology that uses exogenous programmable sequence-specific DNA-binding domains (e.g., engineered zinc fingers (ZFs), transcription activator-like effectors (TALEs), or catalytically inactive RNA-guided CRISPR proteins) to induce targeted endogenous gene regulation. This has been accomplished to date by fusing transcriptional regulatory domains (e.g., transcriptional activation or repression domains) or enzymes that modify histones or DNA (e.g., acetylation and/or methylation enzymes) to these targetable DNA-binding domains and directing them to a target endogenous gene or sequences that can regulate that gene (e.g., promoters and/or enhancers).

Here we describe the surprising finding that ectopic binding of endogenous CTCF (or an engineered variant CTCF (vCTCF) protein with altered DNA-binding specificity) to an endogenous human gene promoter can mediate robust activation of that target gene. This gene activation can be induced in a stable and heritable fashion by using gene editing to introduce an ectopic CTCF binding site (CTCF-BS) into the target promoter, which can then be bound by endogenous CTCF protein. Alternatively, transient activation can be achieved in two different ways using a vCTCF and its associated variant CTCF-BS (vCTCF-BS, also referred to herein as a non-canonical CTCF-BS) either by (1) inserting the vCBS into the target promoter and then expressing the vCTCF transiently or (2) leveraging a vCBS that is already present in the target promoter and transiently expressing a vCTCF that can bind to that vCBS. Although the precise mechanism(s) that mediate this activating effect are not yet fully understood, we also present evidence that the CTCF protein itself may function directly as a transcriptional activator in mammalian cells. See, e.g., U.S. Pat. No. 11,041,155

The present methods can include introducing a CTCF binding site (CTCF-BS) into a promoter region of a target gene, e.g., within 1000, 500, 250, 200, 150, 100, 50, 25, or 10 nucleotides of the TSS for the target gene. In some embodiments, the CTCF-BS comprises “canonical consensus CBS” that contains the following core sequence: 5′-CCAGCAGGGGGCGCT-3′ (SEQ ID NO:1). Alternatively, a variant CTCF-BS can be used with its corresponding non-canonical CTCF, e.g., as described in U.S. Pat. No. 11,041,155; for example, in some embodiments, the non-canonical CTCF-BS comprises one of the following core sequences: 5′-CGAGGAGGGGACGCT-3′ (SEQ ID NO:2), 5′-CAAGCGTGGTGCGCT-3′ (SEQ ID NO: 3), or 5′-CGAGCGTGGTGCGCT-3′ (SEQ ID NO:4). Preferably the CTCF-BS is introduced in the “right” orientation as shown in the figures, i.e., in a 5′ to 3′ direction on the sense strand with respect to the sequence encoding the target gene.

A number of methods known in the art can be used to introduce the CTCF-BS into the target promoter, including gene editing nucleases mediating non-homologous end-joining repair, capture of double-stranded oligonucleotides (dsODNs), or microhomology-mediated repair; prime editing; CRISPR-based editing; base editing; and homologous recombination or homology-directed repair. See, e.g., Liu et al., Mol Cell. 2022 Jan. 20; 82 (2): 333-347; Kantor et al., Int J Mol Sci. 2020 September; 21 (17): 6240; Anzalone et al., Nat Biotechnol. 2020 July; 38 (7): 824-844; and U.S. Pat. Nos. 11,326,157; 11,286,468; 11,220,678; 11,180,751; 11,168,338; 11,168,313; 11,098,326; 11,060,115; 11,060,078; 11,028,429; 11,021,718; 10,894,950; 10,844,403; 10,808,233; 10,800,790; 10,767,168; 10,760,064; 10,738,303; 10,733,354; 10,731, 167; 10,676,749; 10,633,642; 10,587,869; 10,544,433; 10,526,591; 10,526,589; 10,501,794; 10,479,982; 10,417,388; 10,415,059; 10,378,027; 10,273,271; 10,202,589; 10,138,476; 10,119,133; 10,093,910; 10,011,850; 9,988,674; 9,944,912; 9,926,546; 9,926,545; 9,890,364; 9,885,033; 9,850,484; 9,822,407; 9,752,132; 9,567,604; 9,567,603; and 9,512,446.

The present methods can further include expressing in or introducing into the cell the CTCF protein or variant thereof, e.g., using methods known in the art, for stably or transiently expressing the CTCF protein or variant thereof.

Sequences for human CTCF are known in the art; exemplary sequences are shown in Table A. Others are provided in U.S. Pat. No. 11,041,155.

TABLE A
EXEMPLARY HUMAN CTCF SEQUENCES

| | | |
|---|---|---|
| NM_006565.4 | NP_006556.1 | transcriptional repressor CTCF isoform 1* |
| NM_001191022.2 | NP_001177951.1 | transcriptional repressor CTCF isoform 2** |
| NM_001363916.1 | NP_001350845.1 | transcriptional repressor CTCF isoform 3 |

*variant (1) is the longer transcript and encodes the longer isoform (1).
**variant (2) lacks internal two consecutive exons, resulting in a downstream AUG start codon, as compared to variant 1. The resulting isoform (2) has a shorter N-terminus, as compared to isoform 1.

In some embodiments of the methods and compositions described herein, variants of any of the CTCF proteins or nucleic acids described herein can also be used that are at least 80%, 85%, 90%, 95%, 97%, 98%, or 99% identical to a sequence provided herein can also be used, so long as they retain desired functionality of the parental sequence. Residues that can be changed without destroying function can be identified, e.g., by aligning similar sequences and making conservative substitutions in non-conserved regions. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

The present methods can be used in any cell, preferably in mammalian, e.g., human cells. The cells can be primary cells, e.g., in culture, optionally obtained from a human subject, or can be cultured cells, e.g., cell lines. In some embodiments, the cells are induced pluripotent stem cells (iPSCs) or embryonic stem (ES) cells, e.g., human ES (hES cells). Also provided herein are cells that have been altered as described herein to include an exogenous canonical or non-canonical CTCF-BS in the promoter region of a target gene in the cell, e.g., within 1000, 500, 250, 200, 150, 100, 50, 25, or 10 nucleotides of the transcription start site (TSS) for the target gene.

In some embodiments, the cell is heterozygous for the target gene, and the CTCF-BS is specifically directed to be inserted into the promoter of one allele using a gene editing method directed to a SNP in that allele.

### EXAMPLES

The invention is further described in the following examples, which do not limit the scope of the invention described in the claims.

### Materials and Methods

The following materials and methods were used in the examples below.

### Molecular Cloning

The prime editor (PE) construct was from Addgene plasmid (Addgene #112101). All guide RNA (gRNA) constructs were cloned into a BsmBI-digested pUC19-based entry vector (BPK1520, Addgene #65777) with a U6 promoter driving the gRNA expression. We designed the pegRNAs following the previously described default design rules for designing pegRNAs and ngRNAs (Anzalone et al, Nature 2019, 576, pages 149-157). PegRNAs were cloned into the BsaI-digested pU6-pegRNA-GG-acceptor entry vector (Addgene #132777) and ngRNAs were cloned into the BsmBI-digested entry vector BPK1520 that is mentioned above. Oligos containing the spacer, the 5′phosphorylated pegRNA scaffold, and the 3′ extension sequences were annealed to form dsDNA fragments with compatible overhangs and ligated using T4 ligase (NEB). All plasmids used for transfection experiments were prepared using Qiagen Midi or Maxi Plus kits.

PegRNAs and ngRNAs are described in Table B.

TABLE B
PegRNAs and ngRNAs

| Target | CBS | | | |
|---|---|---|---|---|
| promoter | orientation | pegRNA spacer | pegRNA 3′ extension* | ngRNA spacer |
| SGCA | Right | TTTGGTGCATGC | GTCagcgccccctgctgg | CCTCCAACCGTC |
| | | TCCAGGCG | CCTGGAGCATGCA | CCCTCCAG |
| | | (SEQ ID | (SEQ ID NO: 6) | (SEQ ID |
| SGCA | Left | NO: 5) | TGGGTCCCAGCGTCccagcagggggcgctC | NO: 8) |
| | | | CTGGAGCATGCAC (SEQ ID NO: 7) | |
| IL2RA | Right | GGATGAGAGAAG | ATTGGGCTGGCGTGT | GTTGATGACAAT |
| | | AGAGTGCT | TCAGCCAGGAAACTGC | ATAGTTTG |
| | | (SEQ ID | CTAGCccagcagggggcgc | (SEQ ID |
| | | NO: 9) | tACTCTCTTCTCTCA | NO: 12) |
| | | | (SEQ ID NO: 10) | |
| IL2RA | Left | | ATTGGGCTGGCGTGTT | |
| | | | CAGCCAGGAAACTGCC | |
| | | | TAGCagcgccccctgctggA | |
| | | | CTCTCTTCTCTCA (SEQ ID NO: 11) | |
| HER2 | Right | CCCTCTCTTCGC | AGGCGTCCCGGCGCTA | CTGCATTTAGGG |
| | | GCAGGCCT | GGAGGGACGCACCCA | ATTCTCCG |
| | | (SEQ ID | GGccagcagggggcgctC CTGCGCGAAGA | (SEQ ID |
| | | NO: 13) | (SEQ ID NO: 14) | NO: 16) |
| HER2 | Left | | AGGCGTCCCGGCGCT AGGAGGGACGCACC | |
| | | | CAGGagcgccccctgctgg | |
| | | | CCTGCGCGAAGA | |
| | | | (SEQ ID NO: 15) | |
| CD4 | Right | GACATGTTCCCT | GGAGCTGGGTagcgcc | AGCAGAATCAGG |
| | | GAGAGCCT | ccctgctggCTCTCAGGGAACA (SEQ ID | CTTAAATC |
| | | (SEQ ID | NO: 18) | (SEQ ID |
| | | NO: 17) | | NO: 20) |
| CD4 | Left | | ACGTCACCAGCTGGAGC | GGAAAAAGTTAA |
| | | | TGGGTccagcagggggcgc | GCAGAATC |
| | | | tCTCTCAGGGAACATG | (SEQ ID |
| | | | (SEQ ID NO: 19) | NO: 21) |

*lower case: sequence that is modified (CTCF binding sequence); upper case: hybridizing sequence

### Cell Culture and Transfections

STR-authenticated HEK293T (CRL-3216) and K562 (CCL-243) cells were used in this study. HEK293T cells were grown in Dulbecco's Modified Eagle Medium (DMEM, Gibco) with 10% heat-inactivated fetal bovine serum (FBS, Gibco) supplemented with 1% penicillin-streptomycin (Gibco) antibiotic mix. K562 cells were grown in Roswell Park Memorial Institute (RPMI) 1640 Medium (Gibco) with 10% FBS supplemented with 1% Pen-Strep and 1% GlutaMAX (Gibco). Cells were grown at 37° C. in 5% CO2 incubators and periodically passaged upon reaching around 80% confluency. Cell culture media supernatant was tested for mycoplasma contamination using the MycoAlert mycoplasma detection kit (Lonza) and all tests were negative throughout the experiments.

### Transfections

HEK293T cells were seeded at 6.25×10⁴ cells per well into 24-well cell culture plates (Corning). 24 hours post-seeding, cells were transfected with 300 ng prime editor plasmid, 100 ng pegRNA, and 33.2 ng nicking gRNA, and 3 μL TransIT-X2 for experiments in 24-well plates. K562 cells were electroporated using the SF Cell Kit V (Lonza), according to the manufacturer's protocol with 2×10⁵ cells per nucleofection and 800 ng control or prime editor plasmid, 200 ng gRNA or pegRNA plasmid, and 83 ng nicking gRNA plasmid. 72 hours post-transfection, cells were lysed for extraction of genomic DNA (gDNA).

### DNA and RNA Extraction

For DNA on-target experiments in 96-well plates, 72 h post-transfection, cells were washed with PBS, lysed with freshly prepared 43.5 μL DNA lysis buffer (50 mM Tris HCl pH 8.0, 100 mM NaCl, 5 mM EDTA, 0.05% SDS), 5.25 μL Proteinase K (NEB), and 1.25 μL 1M DTT (Sigma). For DNA off-target experiments in 24-well plates, cells were lysed in 174 μL DNA lysis buffer, 21 μL Proteinase K, and 5 μL 1M DTT. For RNA off-target experiments, GFP sorted cells were split 20% for DNA and 80% for RNA extraction. Cells were centrifuged (200 g, 8 min) and lysed as above for DNA extraction or with 350 μL RNA lysis buffer LBP (Macherey-Nagel) for RNA extraction. Lysates for DNA extraction were incubated at 55° C. on a plate shaker overnight, then gDNA was extracted with 2x paramagnetic beads (as previously described), washed 3 times with 70% EtOH, and eluted in 30-80 μL 0.1×EB buffer (Qiagen). RNA lysates were extracted with the NucleoSpin RNA Plus kit (Macherey-Nagel) following the manufacturer's instructions.

### Targeted Amplicon Sequencing

DNA targeted amplicon sequencing was performed as previously described (Grünewald et al, Nature 2019, 569, pages 433-437). Briefly, extracted gDNA was quantified using the Qubit dsDNA HS Assay Kit (Thermo Fisher). Amplicons were constructed in 2 PCR steps. In the first PCR, regions of interest (170-250 bp) were amplified from 5-20 ng of gDNA with primers containing Illumina forward and reverse adapters on both ends. PCR products were quantified on a Synergy HT microplate reader (BioTek) at 485/528 nm using a Quantifluor dsDNA quantification system (Promega), pooled and cleaned with 0.7× paramagnetic beads, as previously described. In a second PCR step (barcoding), unique pairs of Illumina-compatible indexes (equivalent to TruSeq CD indexes, formerly known as TruSeq HT) were added to the amplicons. The amplified products were cleaned up with 0.7× paramagnetic beads, quantified with the Quantifluor or Qubit systems, and pooled before sequencing. The final library was sequenced on an Illumina MiSeq machine using the Miseq Reagent Kit v2 (300 cycles, 2×150 bp, paired-end). Demultiplexed FASTQ files were downloaded from BaseSpace (Illumina).

### Targeted Amplicon Sequencing Analysis

Amplicon sequencing data were analyzed with CRISPResso2 2.0.3016 run in HDR output mode.

### Flow Cytometry

Cells were washed with cell staining buffer (Biolegends) 72 hours post-transfection and incubated with PE conjugated IL2RA (Biolegends), CD4 (Biolegends) HER2 (Biolegends) for 15 minutes, followed by two washes with cell staining buffer. All PE positive cells were measured by a LSR Fortessa X-20 flow cytometer (BD) to test target protein expression.

### Measurement of Target Gene Expression

For target gene expression analysis, total RNA was extracted from the cells 72 hours post-transfection using the NucleoSpin RNA Plus Kit (Clontech, cat #740984.250) and 250 ng of purified RNA was used for cDNA synthesis using a High Capacity RNA-to-cDNA kit (ThermoFisher, cat #4387406). 3 μl of 1:20 diluted cDNA was amplified by quantitative PCR (qPCR) using Fast SYBR Green Master Mix (ThermoFisher, cat #4385612) with the primers listed elsewhere in this application. qPCR reactions were performed on a LightCycler 480 (Roche) with the following program: initial denaturation at 95° C. for 20 seconds(s) followed by 45 cycles of 95° C. for 3 s and 60° C. for 30 s. Ct values greater than 35 were considered as 35, because Ct values fluctuate for transcripts expressed at very low levels. Gene expression levels were normalized to HPRT1 and calculated relative to that of the negative controls (PE3 with non-targeting pegRNA).

### CTCF HiChIP

The HiChIP MNase library was prepared using the Dovetail® HiChIP MNase Kit according to the manufacturer's protocol. Briefly, the chromatin was fixed with disuccinimidyl glutarate (DSG) and formaldehyde in the nucleus. The cross-linked chromatin was digested in situ with micrococcal nuclease (MNase) then extracted upon cell lysis. The chromatin fragments were incubated with the respective antibody overnight for chromatin immunoprecipitation after which, the antibody-protein-DNA complex was pulled down with protein A/G-coated beads. Next, the chromatin ends were repaired and ligated to a biotinylated bridge adapter followed by proximity ligation of adapter-containing ends. In the following steps, the crosslinks were reversed, the associated proteins were degraded, and the DNA was purified and converted into a sequencing library using Illumina-compatible adaptors. Biotin-containing fragments were isolated using streptavidin beads prior to PCR amplification. The library was sequenced on an Illumina Nextseq 2000 platform to generate ˜150 million 2×150 bp read pairs.

### Capture Micro-C

The Micro-C library was prepared using the Dovetail® Micro-C Kit according to the manufacturer's protocol. Briefly, the chromatin was fixed with disuccinimidyl glutarate (DSG) and formaldehyde in the nucleus and the cross-linked chromatin was then digested in situ with micrococcal nuclease (MNase). Next, the cells were lysed with SDS to extract the chromatin fragments which were then bound to Chromatin Capture Beads. The chromatin ends were then repaired and ligated to a biotinylated bridge adapter followed by proximity ligation of adapter-containing ends. In the following steps, the crosslinks were reversed, the associated proteins were degraded, and the DNA was purified then converted into a sequencing library using Illumina-compatible adaptors. Biotin-containing fragments were isolated using streptavidin beads prior to PCR amplification. Capture was performed in accordance with Twist Bioscience's Standard Hybridization Target Enrichment Protocol. Post-capture libraries across samples were pooled in a 1:1 molar ratio. Pooled libraries were sequenced by paired-end 2×150 cycle sequencing kits with Illumina Nextseq2000 system to generate ˜200 M reads per sample.

### Capture Probe Design

The target locus was a 1.5-Mb-sized region centered on SGCA gene. 80-mer probes were designed to tile end-to-end without overlap across the capture loci through Twist Bioscience. Probes with high predicted likelihoods of off-target pull-down (for example, such as those in high-repeat regions) were masked and removed from the probe tiling, and probe coverage was double-checked to ensure the inclusion of key genomic features (for example, de novo CTCF binding sites at the SGCA promoter) before finalization. Probe panels were synthesized and purchased as Custom Target Enrichment Panels from Twist Bioscience.

### Example 1. CTCF-Mediated Activation of the Endogenous Human SGCA Gene In K562 Cells

We first explored whether targeting of endogenous CTCF protein to a promoter of interest could mediate human gene activation. These experiments were driven by our surprising finding that off-target binding of a previously described vCTCF protein to a sequence in the human SGCA promoter led to very robust activation of that gene in human K562 cells (FIGS. 1A and 1B). We next tested whether conversion of this binding site to a consensus CBS that could be bound by endogenously expressed CTCF might also lead to activation of the SGCA gene in K562 cells. Using CRISPR-mediated prime editing, we performed this conversion in two different ways that create a consensus CBS in one orientation or the other (shown as → and ← in FIGS. 1C and 1D and referred to as the “right” and “left” orientations hereafter, respectively; the “right” orientation is 5′→3′ on the “top” or sense strand with respect to the sense strand of the target gene, while the “left” orientation is 5′→3′ on the antisense strand). We isolated single cell K562 clones and screened them for introduction of the consensus CBS, identifying clones for each orientation that showed frequencies of editing that we believe correlate with editing of no, one, two, or three of the three SGCA promoter alleles present in these cells (FIG. 1E). We observed transcriptional activation of the SGCA promoter in all but one of the clones bearing at least one modified allele with more robust activation observed when the consensus CBS was introduced in the “right” relative to the “left” orientation (FIG. 1E).

### Example 2. CTCF-Mediated Activation of the Endogenous Human SGCA Gene In HEK293T Cells

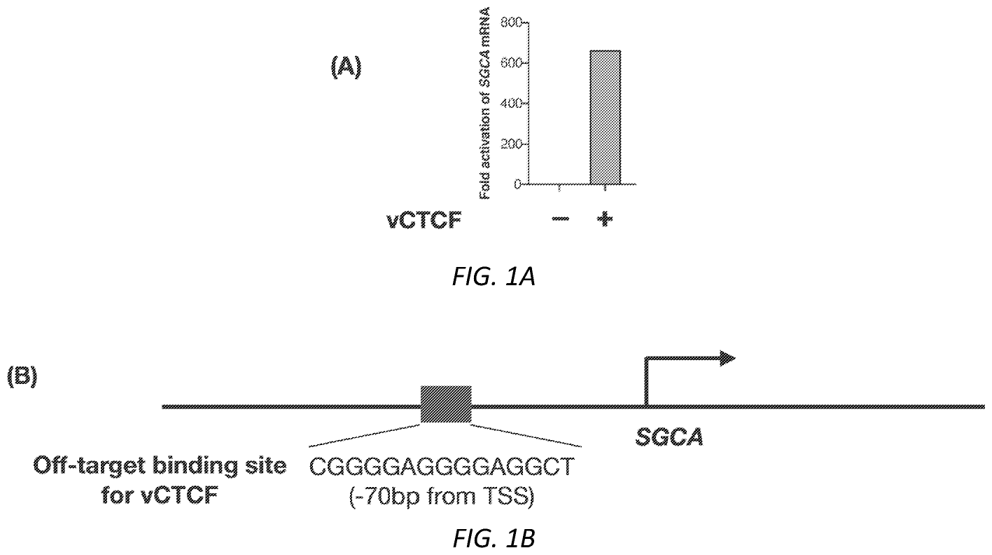

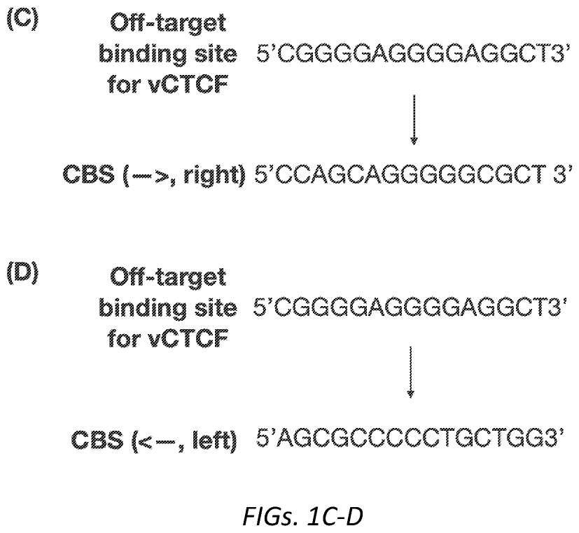

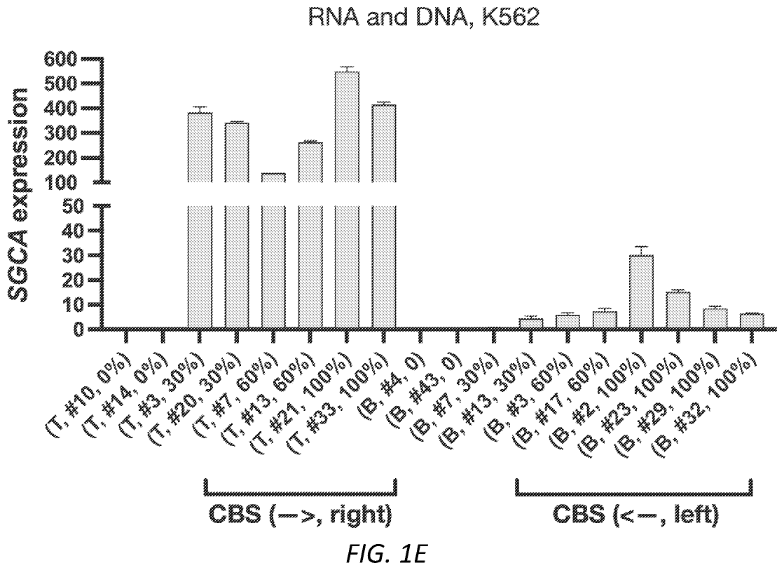

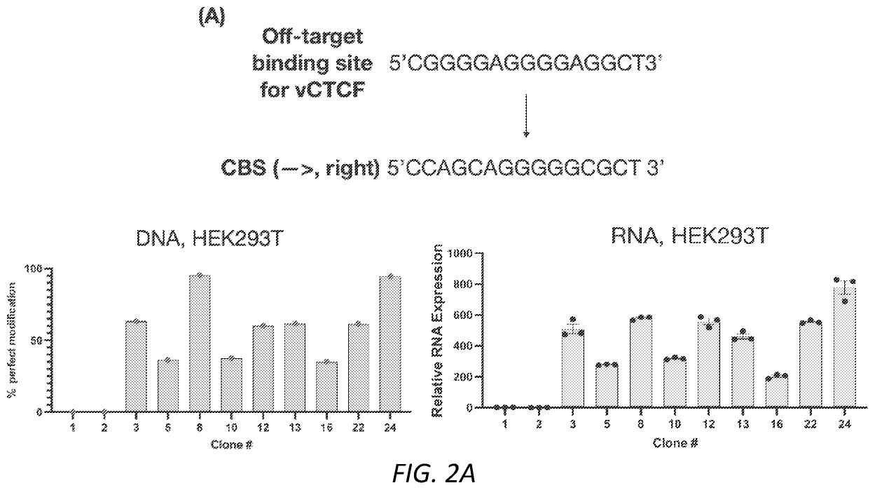

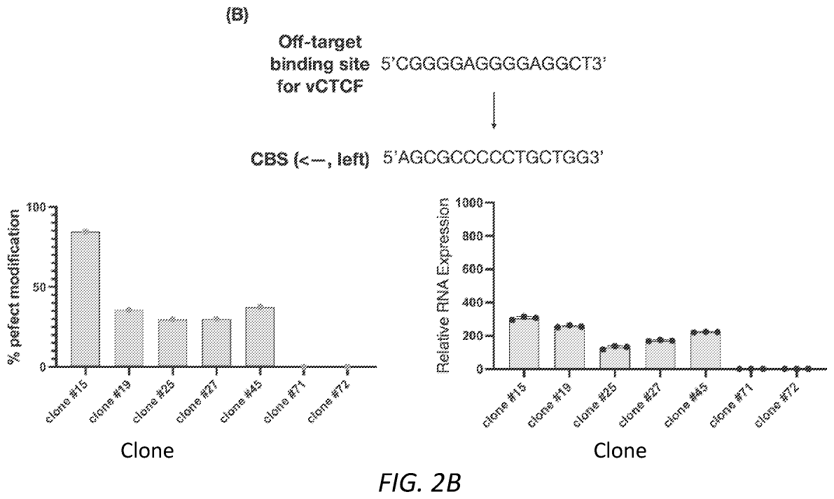

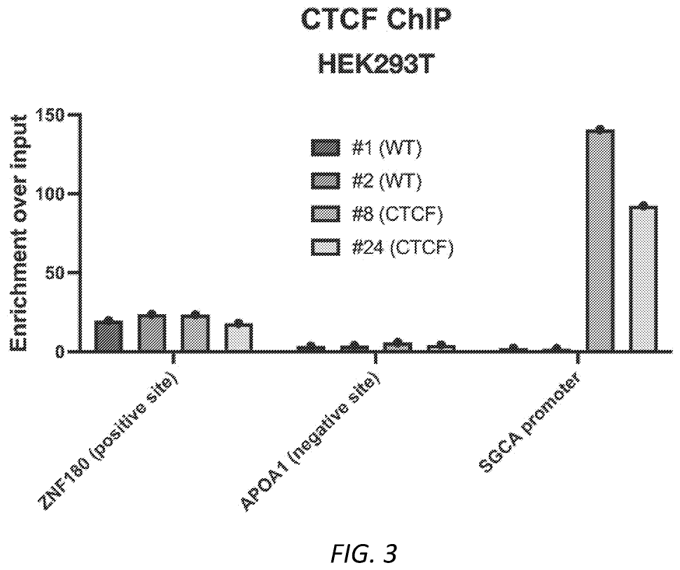

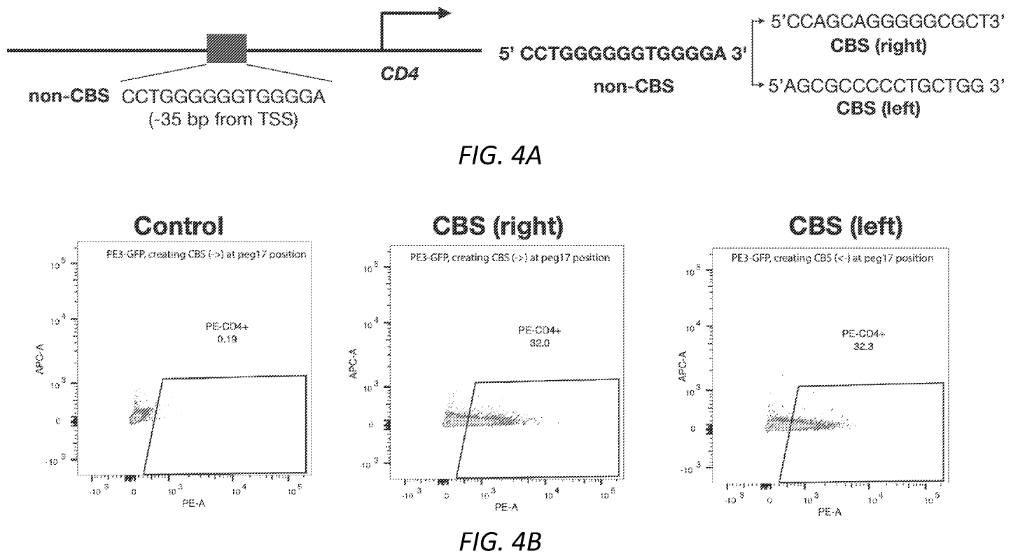

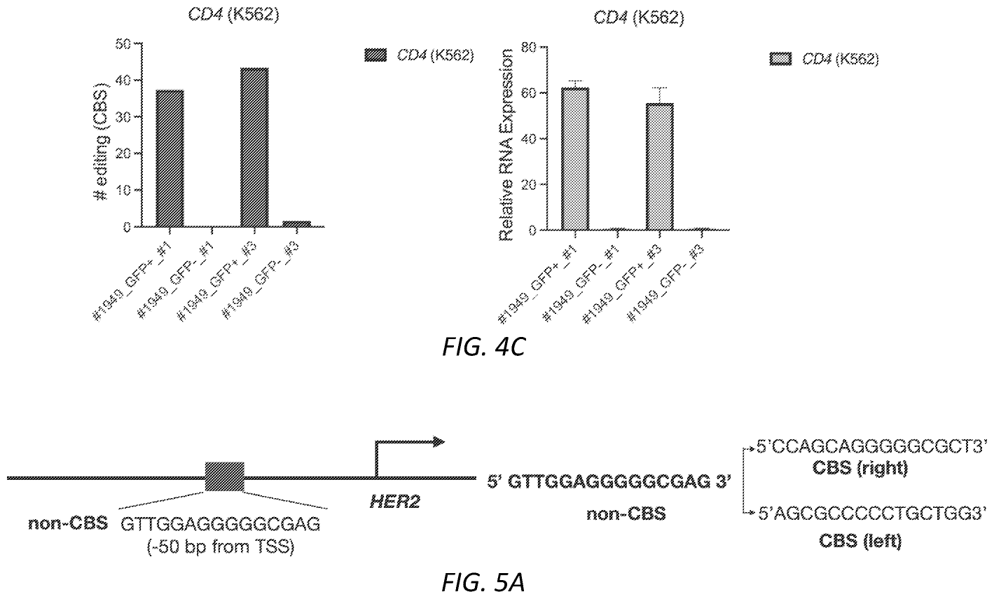

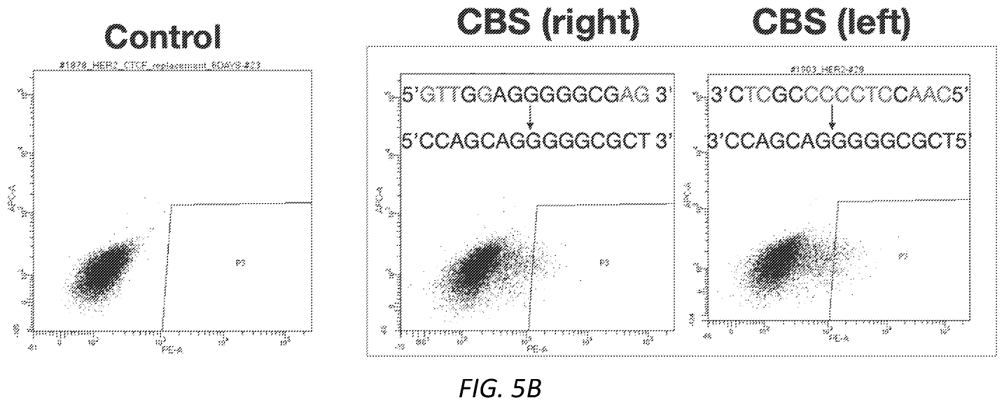

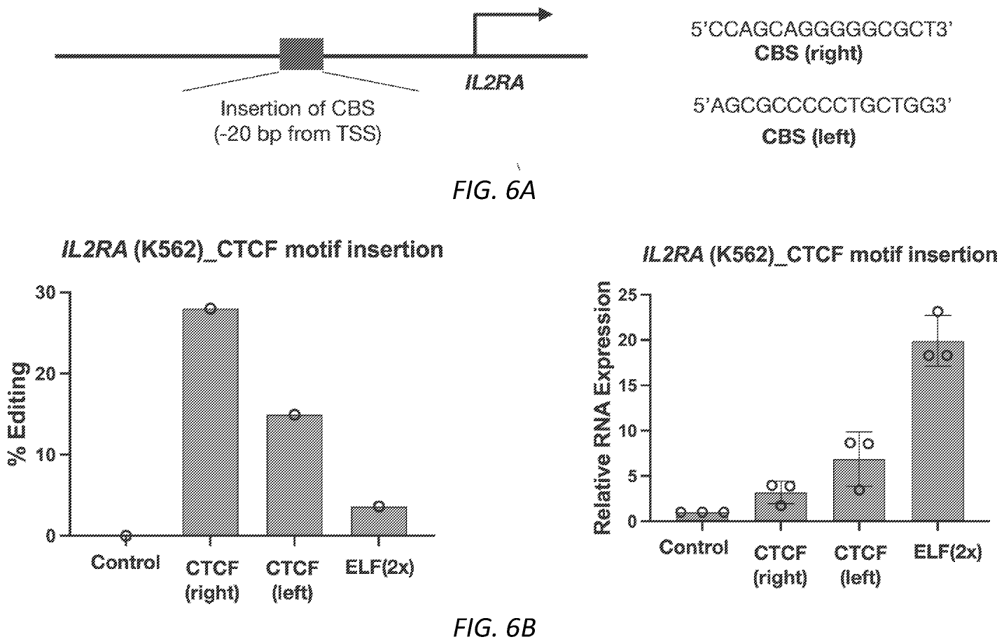

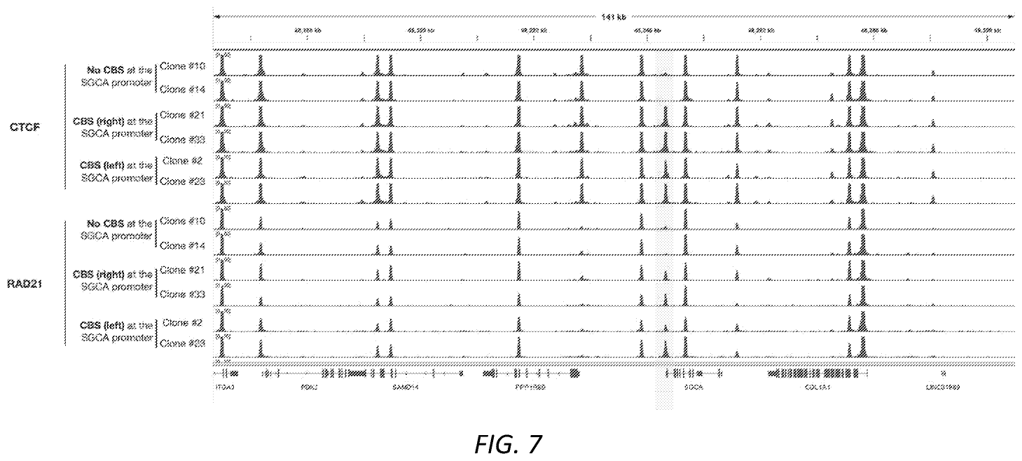

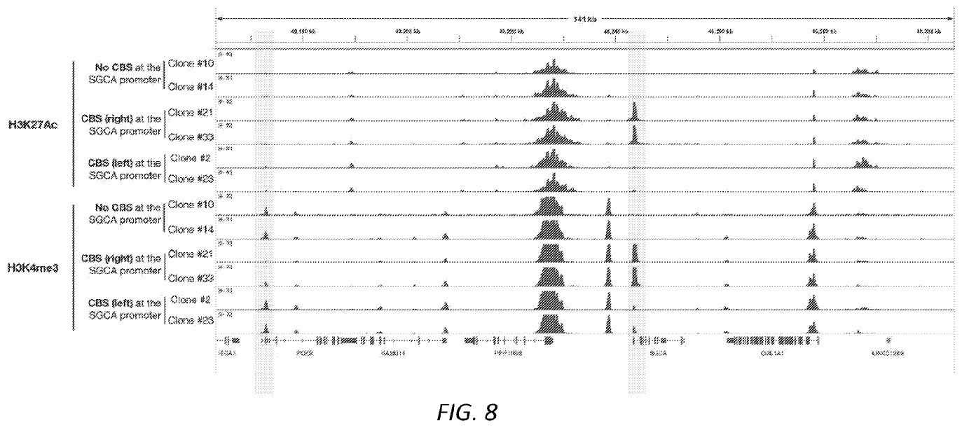

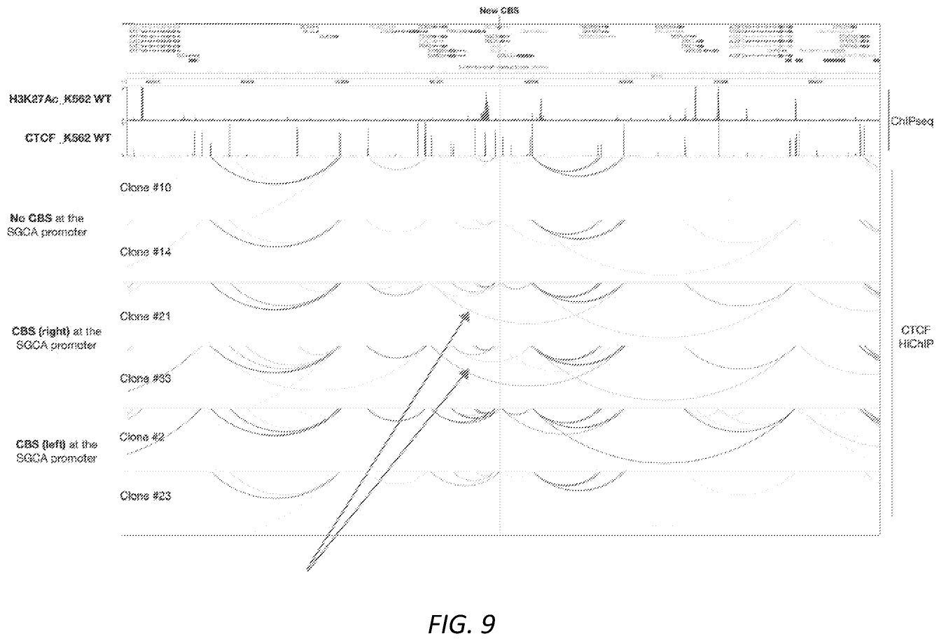

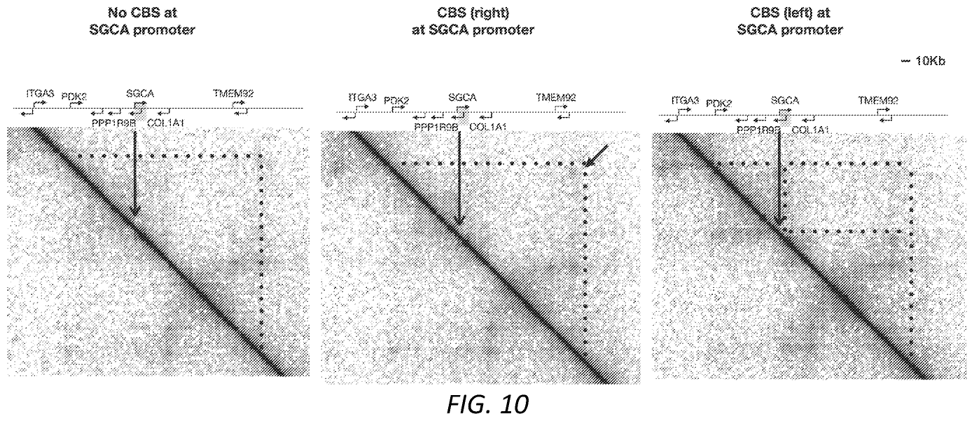

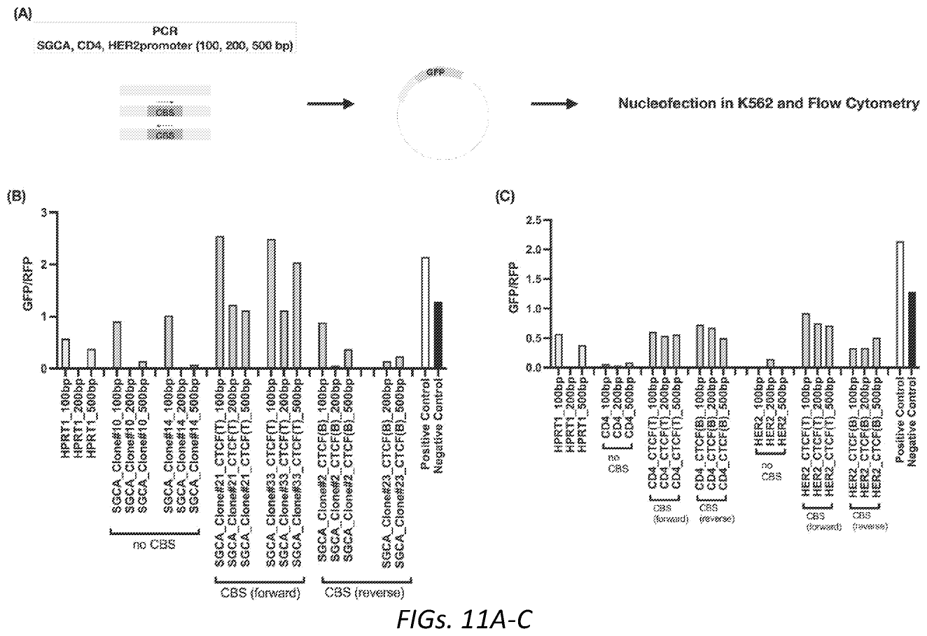

We next tested whether we could similarly activate expression of the SGCA gene in a different human cell line. To do this, we again used CRISPR prime editing to introduce a consensus CTCF site into the SGCA promoter in HEK293T cells in the same “right” and “left” orientations described above (FIGS. 2A and 2B, left panels) and isolated single cell clones that we presume correspond to cells with successful editing of no, one, two, or all three SGCA promoter alleles (FIGS. 2A and 2B, middle panels). Once again, we observed transcriptional activation of the SGCA gene in all clones bearing at least one modified allele (FIGS. 2A and 2B, right panels). Notably, greater activation was again observed with the consensus CBS introduced in the “right” orientation versus the “left” (compare right panels in FIGS. 2A and 2B). In a subset of the HEK293T clones with the consensus CBS in the “right” orientation, we used ChIP-PCR to confirm that endogenous CTCF is bound to this modified sequence in the SGCA promoter (FIG. 3).

### Example 3. Introduction of Consensus CBSs into Additional Human Gene Promoters can Also Induce Gene Activation

To extend the generality of our finding the ectopic CTCF binding in a promoter can lead to transcriptional activation, we used CRISPR prime editing to introduce consensus CBSs into three additional human gene promoters. For the (D) 4, HER2, and IL2RA genes, we identified locations just upstream of the TSS (FIGS. 4A, 5A, and 6A) at which introduction of a consensus CBS site (in either direction—i.e., “right” or “left”) could lead to measurable activation of each of these genes in populations of cells that undergone prime editing as judged by flow cytometry (FIGS. 4B and 5B) and/or by assessment of gene transcripts using quantitative RT-qPCR (FIGS. 4C and 6B). Interestingly, for these three additional genes, we did not observe a striking differential between introduction of the consensus CBS in the “right” and “left” orientations as we did at the SGCA gene.

### Example 4. Exploring the Mechanism of Transcriptional Activation Induced by Ectopic CTCF Binding to a Human Gene Promoter

To begin to delineate the mechanism of CTCF-mediated activation, we sought to perform ChIP-seq with various antibodies to assess the binding of CTCF and RAD21 (a component of the Cohesin complex) and the presence H3K27Ac and H3K4me3 (histone modifications associated with transcriptional activation) at SGCA locus in the six K562 cell clones described above. We performed these experiments using six of the independent K562 cell clones we describe above that had: no introduced consensus CBS (clones #10 and #14), all alleles with the consensus CBS in the “right” orientation (clones #21 and #33), or all alleles with the consensus CBS in the “left” orientation (clones #2 and #23). The results of ChIP-seq demonstrated that both CTCF and RAD21 binding could be detected comparably in all of the cell clones that had the consensus CBS introduced in either orientation but not in the cell clones that did not bear this edit (FIG. 7). Consistent with the degree of SGCA activation we had observed in these cell clones, we also found strong H3K27Ac and H3K4me3 histone modifications at the SGCA promoter only in the clones in which the consensus CBS was introduced in the “right” orientation (FIG. 8). We could observe weak signal for both of these histone modifications in cell clones with the consensus CBS in the “left” orientation but did not see these in the clones lacking the consensus CBS site (FIG. 8).

We hypothesized that at least some of the CTCF-induced activation of the SGCA gene we observed might be due to changes on 3-D architecture at this locus induced by ectopic CTCF binding to the consensus CBS we introduced. To test this, we performed Hi-ChIP experiments using an antibody against CTCF on the same six K562 cell clones we used for the ChIP-seq experiments described above. Analysis of these data revealed the induction of a novel interaction between two genomic sites flanking the TMEM92 gene and PDK2 gene that are separated by ˜201 Kb in the cell clones with the consensus CBS introduced in the “right” orientation (FIG. 9, arrows). Notably, this interaction was not detected in cell clones with no consensus CBS edit or in those in which the consensus CBS was introduced in the “left” orientation (FIG. 9). These data demonstrate an association and suggest a potential causal link between this novel genomic interaction and the observed robust activation of the SGCA gene observed in cell clones bearing the consensus CBS in the “right” orientation.

To identify additional novel interactions at the SGCA locus due to the introduction of CBS, we performed capture Micro-C that captures all-to-all interactions in 1.5 Mb window centered on SGCA promoter. Analysis of this data revealed the strength of TAD structure present at SGCA locus was increased with the introduction of CBS in the “right” orientation at the SGCA promoter. In contrast, CBS with the “left” orientation at the SGCA promoter strengthened the sub TAD structures under the original TAD structure (FIG. 10). This analysis also showed the loop that was previously identified in CTCF HiChIP analysis, which was specific to the clones where CBS was introduced in the “right” orientation (FIG. 10).

We also considered the possibility that CTCF might also be functioning directly as a transcriptional activator when bound ectopically to promoter sequences. To test this possibility, we cloned genomic promoter fragments of various lengths (harboring 100, 200, and 500 bps of sequence upstream of the TSS) from the SGCA, CD4, and HER2 genes that harbor no edit or introduction of the consensus CBS in the “right” or “left” orientations (FIG. 11A). We inserted these fragments upstream of a GFP reporter gene to create a series of different reporter plasmids (FIG. 11A). We then transfected each of these plasmids together with a plasmid that constitutively expresses a red fluorescent protein to control for transfection efficiencies into K562 cells and determined the ratio of GFP to RFP signal using flow cytometry for each sample. The results of the transient transfection experiments revealed reporter gene activation with each of the SGCA promoter fragments harboring the consensus CBS introduced in the “right” orientation relative to the matched wild-type SGCA promoter fragments (FIG. 11B). We observed little to no activation with SGCA promoter fragments with the consensus CBS introduced in the “left” orientation (FIG. 11B). For the CD4 and HER2 promoter fragments, we also observed activation of the reporter gene with the consensus CBS inserted in both orientations relative to matched wild-type promoter fragments (FIG. 11C). These results show the same patterns of relative activation observed at the endogenous gene promoters with the consensus CBS introduced in the two different orientations. However, because our reporter plasmids are presumably not chromatinized, these results suggest that endogenous CTCF can function directly as an activator of transcription in human cells. To our knowledge, no study has previously demonstrated that CTCF can function as a transcriptional activator. Taken together with our studies described above examining 3-D genome architecture, our overall results suggest that CTCF may mediate activation both by modifying genomic topology as well as a direct activator of transcription.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of increasing expression of a target gene in a cell, the method comprising introducing a canonical CCCTC-binding factor (CTCF) binding site (CTCF-BS) into a promoter region of the target gene in the cell, preferably within 1000, 500, 250, 200, 150, 100, 50, 25, or 10 nucleotides of the transcription start site (TSS) for the target gene.

2. The method of claim 1, wherein the canonical CTCF-BS comprises the following core sequence: 5′-CCAGCAGGGGGCGCT-3′ (SEQ ID NO:1).

3. The method of claim 1, wherein the canonical CTCF-BS is introduced in the sense strand with respect to the target gene.

4. The method of claim 1, wherein the CTCF-BS is introduced into the target promoter using gene editing nucleases mediating non-homologous end-joining repair, capture of double-stranded oligonucleotides (dsODNs), or microhomology-mediated repair; prime editing; CRISPR-based editing; base editing; and homologous recombination or homology-directed repair.

5. The method of claim 1, wherein the cell expresses a CTCF protein, optionally an endogenous CTCF protein.

6. The method of claim 1, comprising expressing in or introducing into the cell the CTCF protein.

7. A method of increasing expression of a target gene in a cell, the method comprising introducing a non-canonical CCCTC-binding factor (CTCF) binding site (CTCF-BS) into a promoter region of the target gene in the cell, preferably within 1000, 500, 250, 200, 150, 100, 50, 25, or 10 nucleotides of the transcription start site (TSS) for the target gene, and expressing in or introducing into the cell a variant CTCF protein with an altered DNA-binding specificity that binds the non-canonical CTCF-BS.

8. The method of claim 7, wherein the non-canonical CTCF-BS comprises one of the following core sequences: 5′-CGAGGAGGGGACGCT-3′ (SEQ ID NO:2), 5′-CAAGCGTGGTGCGCT-3′ (SEQ ID NO:3), or 5′-CGAGCGTGGTGCGCT-3′ (SEQ ID NO: 4).

9. The method of claim 7, wherein the non-canonical CTCF-BS is introduced in the sense strand with respect to the target gene.

10. The method of claim 7, wherein the non-canonical CTCF-BS is introduced into the target promoter using gene editing nucleases mediating non-homologous end-joining repair, capture of double-stranded oligonucleotides (dsODNs), or microhomology-mediated repair; prime editing; CRISPR-based editing; base editing; and homologous recombination or homology-directed repair.

11. The method of claim 1, wherein the cell is in vitro.

12. The method of claim 1, wherein the cell is in a living animal, e.g., a mammal.

13. An isolated cell comprising an exogenous canonical or non-canonical CCCTC-binding factor (CTCF) binding site (CTCF-BS) in a promoter region of a target gene in a cell, wherein expression of the target gene is increased with respect to a cell of the same type that does not comprise an exogenous CTCF-BS in the promoter region.

14. The isolated cell of claim 13, wherein the exogenous canonical or non-canonical CTCF-BS is within 1000, 500, 250, 200, 150, 100, 50, 25, or 10 nucleotides of the transcription start site (TSS) for the target gene.

15. The isolated cell of claim 13, which expresses an endogenous CTCF that binds the canonical CTCF-BS or a variant CTCF protein with an altered DNA-binding specificity that binds the non-canonical CTCF-BS.

16. The isolated cell of claim 13, wherein the canonical CTCF-BS comprises the sequence: 5′-CCAGCAGGGGGCGCT-3′ (SEQ ID NO:1), or the non-canonical CTCF-BS comprises one of: 5′-CGAGGAGGGGACGCT-3′ (SEQ ID NO:2), 5′-CAAGCGTGGTGCGCT-3′ (SEQ ID NO:3), or 5′-CGAGCGTGGTGCGCT-3′ (SEQ ID NO: 4).

17. The isolated cell of claim 13, wherein the exogenous canonical or non-canonical CTCF-BS is present in the sense strand with respect to the target gene.

18. The isolated cell of claim 13, wherein the CTCF-BS is introduced into the target promoter using gene editing nucleases mediating non-homologous end-joining repair, capture of double-stranded oligonucleotides (dsODNs), or microhomology-mediated repair; prime editing; CRISPR-based editing; base editing; and homologous recombination or homology-directed repair.

19. The isolated cell of claim 13, wherein the cell is in vitro.

20. The isolated cell of claim 13, wherein the cell is in a living animal, e.g., a mammal.
